(19) United States
(12) Patent Application Publication
(10) Pub. No.: US 2024/0423589 A1
(43) Pub. Date: Dec. 26, 2024

(54) APPARATUS FOR VOLUMETRIC ULTRASOUND SCANNING

(71) Applicant: Rivanna Medical, Inc., Charlottesvile, VA (US)

(72) Inventors: Robert Rickel, Charlottesville, VA (US); Nathan Sauder, Barboursville, VA (US); Alexis Nunn, Blue Ridge, GA (US); Andong Liang, Charlottesville, VA (US); Zachary Leonard, Charlottesville, VA (US); Paul Sheeran, Charlottesville, VA (US); Adam Dixon, Charlottesville, VA (US); Frank William Mauldin, Troy, VA (US)

(21) Appl. No.: 18/822,357

(22) Filed: Sep. 2, 2024

Related U.S. Application Data
(63) Continuation of application No. 18/371,027, filed on Sep. 21, 2023.
(60) Provisional application No. 63/536,181, filed on Sep. 1, 2023.
(60) Provisional application No. 63/408,490, filed on Sep. 21, 2022.

(51) Int. Cl. A61B 8/00

(52) U.S. Cl. CPC A61B 8/4281

(57) ABSTRACT

An apparatus for acoustically coupling energy from one or more ultrasound arrays for use in medicine during ultrasound imaging procedures and an ultrasound imaging and medical instrument guidance system that interfaces to said apparatus for the purpose of ultrasound data collection.

### CROSS-REFERENCE TO RELATED APPLICATIONS

The present application relies on the disclosures of and claims priority to and the benefit of the filing date of U.S. application Ser. No. 18/371,027, filed Sep. 21, 2023, which relies on the disclosures of and claims priority to U.S. Application No. 63/408,490, filed Sep. 21, 2022. The present application also relies on the disclosures of and claims priority to and the benefit of the filing date of U.S. Application No. 63/536, 181, filed Sep. 1, 2023.

The disclosures of the above applications are hereby incorporated by reference herein in their entireties.

### STATEMENT REGARDING FEDERALLY SPONSORED RESEARCH

This invention was made with government support under Contract No. 75A50121C00035 awarded by HHS/ASPR/BARDA. The government has certain rights in the invention.

### TECHNICAL FIELD

The present invention is related to ultrasound imaging for the purposes of medicine. More specifically, the invention describes both an apparatus that may be used to acoustically couple an ultrasound transducer to a scanning interface, and an imaging system that interfaces to said apparatus for the purpose of ultrasound data collection.

### BACKGROUND OF THE INVENTION

Ultrasound scanning may be used in medical imaging to detect and diagnose pathology in soft tissues or bony anatomy. Ultrasound image data may be collected as individual two-dimensional frames, a sequence of two-dimensional frames (a time-series, for example), individual three-dimensional volumes, a sequence of three-dimensional volumes, or some combination thereof. The varying embodiments of three-dimensional image data acquisition are commonly referred to as volumetric imaging. The approaches to acquiring volumetric ultrasound data generally consist of redirecting energy from ultrasound arrays either electronically or mechanically in order to transmit and receive information that covers an anatomical volume of interest. Volumetric ultrasound scanning systems produced by different manufacturers may integrate ultrasound imaging arrays having varying formats. These include ‘1D’ arrays that have a single row of a number of transmit/receive elements, ‘1.25D’ and ‘1.5D’ arrays that have two to three rows of a number transmit/receive elements that are electronically configured to generate images along the centerline of the array, or ‘2D’ arrays that contain many rows of a number of transmit/receive elements that can be electronically configured to transmit/receive at arbitrary points in a volume, including off-centerline locations. 2D arrays are inherently capable of producing volumetric data directly, but have significant electrical complexity, are expensive, and typically cover only a limited volumetric span. Accordingly, an improvement is needed over existing art.

Manufacturers developing volumetric scanners that employ 1D arrays, 1.25D arrays, or 1.5D arrays, which only create 2D ultrasound images while fixed at a desired spatial location, most often make use of techniques such as mechanical translation and/or rotation of the array to acquire volumetric datasets. In that approach, the individual 2D images generated by the array at each unique spatial location are recorded and stored in hardware memory as a sub-component of the total volumetric dataset. This approach accommodates volumetric scanning over large surface areas while maintaining relatively low electrical complexity.

To acoustically couple energy from an ultrasound array into patient tissue while the array is mechanically translated and/or rotated, one of several state-of-the-art approaches is typically employed:
(1) The ultrasound array is directly coupled to the patient tissue with acoustic gel spread along the surface of the tissue. This approach has low complexity and low cost, but maintaining optimal coupling and geometric precision along the complex tissue surface while the array translates is challenging.
(2) The ultrasound array is coupled to a rigid acoustically transmissive material (e.g., a plastic lens) via a thin fluid layer by embedding the array inside a fluid-filled chamber enclosed on one end by the rigid acoustically transmissive material. The rigid acoustically transmissive material is then coupled to the patient tissue directly via acoustic gel. This approach has moderate complexity, is non-modular (the sealed fluid chamber and rigid material are maintained in place for the lifetime of the probe) and provides reproducible image quality and geometric precision because the array is not translated along the surface of the tissue directly and the travel path is defined by the inner surface of the rigid material by design. However, coupling via a fluid-filled chamber is not typically employed over large surfaces for standard volumetric imaging applications due to increased weight and challenging serviceability. Furthermore, the rigid structure of the plastic lens limits its ability to conform to irregularly shaped anatomies, which limits volumetric scanning to relatively soft anatomical structures, like the abdomen or breast.
(3) The ultrasound array is coupled to a single-use consumable apparatus containing a flexible sheet of acoustically transmissive material that is impregnated with an aqueous couplant (e.g., acoustic gel). The flexible sheet conforms to the patient anatomy while the array translates and/or rotates along the sheet and adjusts height to remain coupled along the change in patient anatomy. This apparatus is low complexity and modular but requires increased system complexity to track changes in transducer location as it is displaced by patient anatomy. This approach imposes additional operator workflow burden to replace the consumable coupling apparatus prior to imaging a new patient, as aqueous coupling material evaporates over time.

### SUMMARY OF THE INVENTION

The present invention as described herein includes an apparatus that interfaces to an ultrasound imaging system and at least one ultrasound transducer array to enable volumetric ultrasound imaging over large surface areas. In embodiments, the apparatus, which can be a low-cost/low-complexity modular unit that can be serviced or replaced periodically, conforms to the patient's body while maintaining substantial contact between the ultrasound array and an acoustically transmissive material via a layer of, in aspects, non-aqueous media, removing the need for a fluid-filled chamber and eliminating dehydration involved with aqueous acoustic couplants. In embodiments, the invention further describes an ultrasound imaging system that incorporates the apparatus to acquire medical imaging data. Various preferred embodiments of the invention are described herein.

Example embodiments described herein have innovative features, no single one of which is indispensable or solely responsible for their desirable attributes. The following description and drawings set forth certain illustrative implementations of the disclosure in detail, which are indicative of several exemplary ways in which the various principles of the disclosure may be carried out. The illustrative examples, however, are not exhaustive of the many possible embodiments of the disclosure. Without limiting the scope of the claims, some of the advantageous features will now be summarized. Other objects, advantages and novel features of the disclosure will be set forth in the following detailed description of the disclosure when considered in conjunction with the drawings, which are intended to illustrate, not limit, the invention.

In embodiments, the present invention overcomes limitations of approaches employed by commercially available imaging devices by acoustically coupling energy from an ultrasound imaging system into a patient, patient anatomy, or patient tissue (e.g., a receiving body and/or a surface of a receiving body) through a modular, conformable, serviceable and replaceable apparatus. In embodiments, the apparatus (e.g., acoustic coupling article) couples to at least one ultrasound transducer array using non-aqueous media and with a design that eliminates the need for a fluid-filled chamber and enables scanning over large surface areas along a path defined by a rigid or mostly rigid component of the apparatus. The apparatus interfaces with an ultrasound imaging system that includes at least one ultrasound transducer array, along with computer-controlled motorized translation and/or rotation actuators that move the array across the apparatus's transmissive acoustic surface and collect image data. The apparatus can be designed to preserve image quality over tens or hundreds of scanning events prior to service or replacement, although more or less usage events are envisioned before service or replacement. Through computer-readable instructions encoded in memory, for example, the system can be capable of executing routines that detect the need for servicing events (or replacement) related to the modular apparatus.

### BRIEF DESCRIPTION OF THE DRAWINGS

The accompanying drawings illustrate certain aspects of some of the embodiments of the present invention and should not be used to limit or define the invention. Together with the written description the drawings serve to explain certain principles of the invention. For a fuller understanding of the nature and advantages of the present technology, reference is made to the following detailed description of preferred embodiments and in connection with the accompanying drawings, in which:

FIGS. 1A-1B are schematic illustrations of exemplary acoustic coupling articles that couple ultrasound energy from acoustic arrays into acoustic transmissive materials.

FIG. 2 depicts the apparatus in an exemplary embodiment that uses a porous material to retain non-aqueous mobile phase and redistribute the mobile phase as the array translates within the apparatus.

FIG. 3 is a schematic illustration of an acoustic coupling article incorporated into a modular frame that clips into the housing of an ultrasound imaging system probe.

FIG. 4 depicts a flow diagram of a process by which an ultrasound imaging system interacts with the coupling apparatus for the purpose of detecting and automating servicing events.

FIG. 5 depicts an acoustic coupling article incorporated into an ultrasound imaging system probe that contains mechanical actuators for translating or rotating ultrasound transducer arrays to collect ultrasound data at varied spatial positions.

FIG. 6A-B depicts an exemplary ultrasound imaging system integrating ultrasound probes and embodiments of the acoustic coupling articles for coupling acoustic energy from ultrasound transducer arrays into patient anatomies. The ultrasound imaging system incorporates structural elements for supporting and positioning ultrasound probes and patient anatomies during ultrasound scanning.

FIG. 7A-B depict example ultrasound probes and patient positions during scanning with an embodiment of the invention.

FIG. 8 depicts an additional embodiment of an acoustic coupling article with a concave surface for interfacing to particular patient anatomies, along with fiducial markers that assist with alignment of patient anatomies with the scanning field of view of the ultrasound probe.

### DETAILED DESCRIPTION OF VARIOUS EMBODIMENTS OF THE INVENTION

The present disclosure describes various systems and methods for constructing and utilizing a modular apparatus containing a transmissive acoustic interface that couples between a patient's body and an ultrasound imaging and medical instrument guidance system comprising at least one ultrasound transducer array. The present disclosure can be used in medical ultrasound applications but is not limited to this application. Those skilled in the art will appreciate that a variety of types of transducers, signal transmitters and/or receivers and other arrays can also benefit from the present invention, which are comprehended hereby. The preferred embodiments herein describe volumetric anatomical imaging. Those skilled in the art will appreciate that the present invention may be used to acquire and process a variety of types of ultrasound image data acquisition including, but not limited to, B-mode, Doppler/PW/CW/flow imaging, contrast imaging, tissue elasticity imaging, and tissue characterization imaging. Furthermore, those skilled in the art will appreciate that the present invention may also be applied to use cases other than anatomical imaging, including, but not limited to, interventional procedure guidance, therapeutic ultrasound guidance, surgical guidance, and pre-surgical planning. The present invention can be utilized, in a preferred embodiment, with an apparatus previously disclosed by Mauldin et al. (U.S. Appl. No. 63/408,490) for conveying ultrasonic energy, which is incorporated by reference herein.

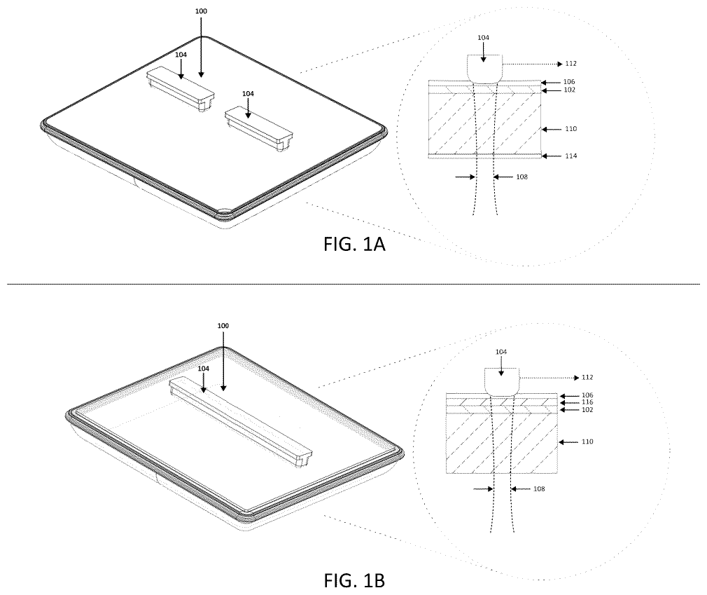

In an embodiment, the acoustic coupling article 100 is depicted in FIG. 1A. In the depicted embodiment, the device can have a single rigid acoustic transmissive material 102, preferably comprising a material such as a plastic, that acoustically couples to one or more ultrasound transducer arrays 104 via a non-aqueous mobile phase 106, preferably comprised of a petroleum, synthetic, or silicone based lubricant. In aspects, acoustic energy is emitted and received through the pathway defined by the non-aqueous fluid 106 and acoustic transmissive material 102 along a spatial extent 108 of the ultrasound beam defined by the ultrasound transducer array geometry. In another preferred embodiment, substance 110 may be a polyurethane-based coupling apparatus previously disclosed by Mauldin et al. (U.S. Appl. No. 63/408,490). In another embodiment, an additional layer 114 encapsulates substance 110, providing protection against environmental damage and is resistant to moisture, UV radiation, chemical exposure and mechanical damage such as cutting, tearing, or abrasion. In another embodiment, the additional layer 114 is the patient contact surface and has been modified by the addition of a surface coating that increases the hydrophilicity, lubricity, and wettability of layer 114. (In other embodiments, the layer 110 can directly contact the patient or a surface of a patient's anatomy (see, e.g., FIG. 1B).) In the preferred embodiment, the material of layer 114 is a thin polyurethane film but may also be comprised of a silicone rubber film or thermoplastic film, such as a polyether block amide (i.e. PEBAX) or polymethylpentene. The ultrasound transducer array 104 translates or rotates along the surface of the acoustic transmissive material 102 such that the path of motion 112 is constrained to be along the surface of the rigid acoustic transmissive material 102. The acoustic transmissive material 102 may be designed to have a surface geometry, such as a flat planar surface, or may be concave, convex, or an otherwise complex shape or texture so as to provide a constrained path of motion for the ultrasound transducer array 104.

In an embodiment, the acoustic coupling article 100 is depicted in FIG. 1B to have a second acoustic transmissive material 116, preferably comprising a polyurethane or silicone material, placed in between the rigid acoustic transmissive material 102 and a layer of non-aqueous mobile phase 106 so as to provide a semi-deformable substance that compresses upon pressure from the ultrasound transducer array and enhances acoustic coupling via improved spreading of the non-aqueous mobile phase 106 along the surface of the ultrasound transducer array 104. In a preferred embodiment, the second acoustic transmissive material 116 may be designed to absorb the non-aqueous mobile phase 106 or to be impregnated with the non-aqueous mobile phase 106 so as to release the non-aqueous mobile phase in response to compression or contact by the ultrasound array 104 so as to improve acoustic coupling.

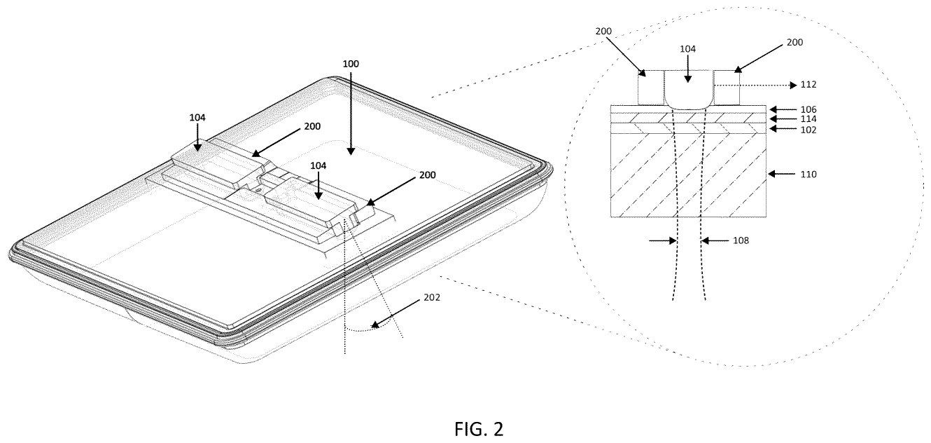

In an embodiment, depicted in FIG. 2, the acoustic coupling article 100 includes a porous material 200, such as a sponge or foam, that can be constructed to conform around one or more of the non-imaging sides of the ultrasound transducer array 104 while simultaneously making contact with a surface of the acoustic coupling article 100. The porous material 200 can be, in aspects, saturated with the non-aqueous mobile phase 106 and contact the rigid acoustic transmissive material 102 so as to spread or distribute the non-aqueous mobile phase 106 along the surface of the acoustic transmissive material 102 as the ultrasound transducer array 104 translates and or rotates during image acquisition. In this embodiment, the porous material 200 effectively serves as a reservoir of the non-aqueous mobile phase 106 that collects and re-applies the non-aqueous mobile phase along the path of motion of the ultrasound transducer array 104. In additional embodiments, the ultrasound transducer array 104 may be angled relative to the surface of the acoustic coupling article 100, as depicted by angle 202. As will be appreciated by those skilled in the art, the ability to angle the ultrasound transducer array 104 relative to the surface acoustic coupling article 100 serves to reduce the amplitude of acoustic reverberations that result from acoustic waves incident on parallel interfaces.

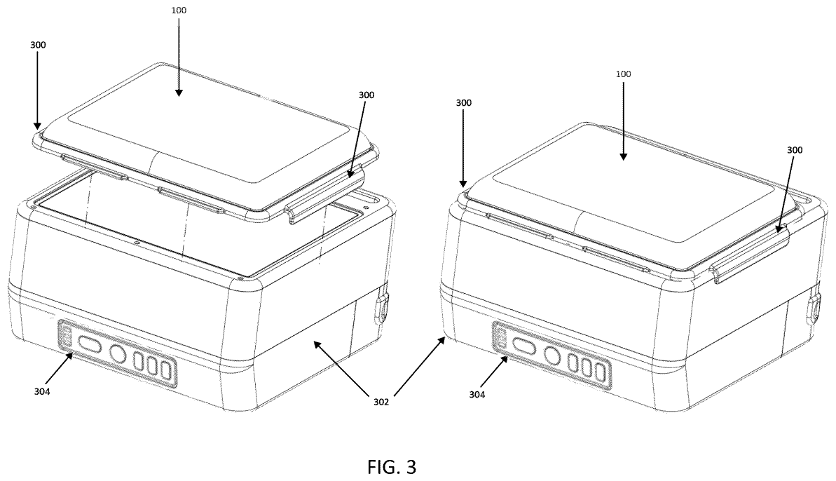

In a preferred embodiment depicted in FIG. 3, the body of the acoustic coupling article 100 incorporates a structural frame 300 that interfaces with an ultrasound imaging probe 302 having one or more ultrasound transducer arrays 104. The structural frame 300 may incorporate retention features for the purpose of mating and durably attaching the acoustic coupling article 100 to the ultrasound imaging probe 302 during use. In a preferred embodiment, the retention features comprise snaps, grooves, clips, or other mechanical fixturing elements that allow the acoustic coupling article 100 to be detached from the ultrasound imaging probe 302. It will be appreciated by those skilled in the art that the inclusion of a detachable mechanism enables the replacement, repair, and maintenance of both the acoustic coupling article 100 and the internal mechanisms within the ultrasound imaging probe 302. In another preferred embodiment, the acoustic coupling article 100 incorporates a fluid-tight seal that is positioned at the physical interface between the acoustic coupling article 100 and the ultrasound imaging probe 302, the seal comprising a gasket, O-ring, or sealant and structural elements including grooves, mating surfaces, or alignment features. Those skilled in the art will recognize that the inclusion of a fluid-tight seal both prevents fluids and contaminants from entering the internals of the ultrasound imaging probe 302 while also preventing leakage of internal fluids, such as the non-aqueous mobile phase 106. In another preferred embodiment, a user interface element 304 may be incorporated to provide control over the ultrasound imaging probe 302 and ultrasound imaging system 600, report probe/system state information, and report probe/system error information. Those skilled in the art will appreciate that the incorporation of integrated user interface controls on the ultrasound probe 302 provides the user with the ability to control the ultrasound imaging probe 302 while also holding the probe against the patient's body, without the need to use a separate user interface, for example, a separate touchscreen or keyboard in the ultrasound imaging system 600.

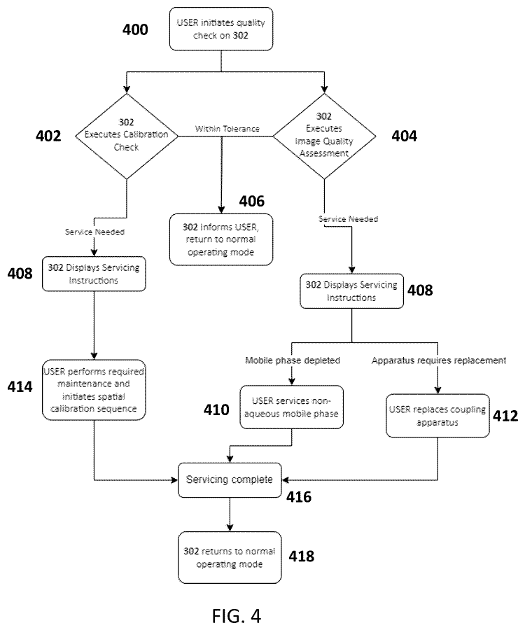

FIG. 4 illustrates a flow diagram of an embodiment of the present invention, enabled in part by the detachable acoustic coupling article 100. The process includes the user initiating a diagnostic check, also known as a quality check, via a graphical user interface on the ultrasound imaging system 600 or ultrasound probe 302 (block 400). The ultrasound imaging system 600 or ultrasound probe 302 performs diagnostic routines, including an automated image quality assessment (block 402) and a geometric calibration (block 404) to evaluate the operational state of the system. If diagnostics indicate that servicing is necessary, then the system displays servicing instructions on a graphical display (block 408). Potential servicing actions include the reapplication of non-aqueous mobile phase 106 to the acoustic coupling article 100 (block 410), removing and replacing the acoustic coupling article 100 (block 412), or initiating a geometric calibration sequence of the ultrasound probe 302 (block 414). Upon completion of servicing, the diagnostic process notifies the user that servicing is complete (block 416) and the system returns to normal operation (block 418). Those skilled in the art will appreciate that this diagnostic process leverages the detachability of the acoustic coupling article 100 from the ultrasound probe 302 to facilitate system maintenance and maintain optimal imaging performance. In a preferred embodiment, the image quality assessment may take the form of one or more analytical algorithms that monitor for various performance issues, including monitoring for degraded data quality due to poor acoustic coupling through acoustic coupling article 100, which can manifest as acoustic dropout or shadowing. In other cases, the algorithm may also monitor for aberrant electrical performance, which may result from electrical interference, damaged sensors on the ultrasound transducer array, or compromised electronic connectors or internal electronics in the ultrasound imaging system 600. This list of diagnostic checks is not exhaustive and those skilled in the art will appreciate that other performance metrics for an ultrasound imaging system 600 may be automatically evaluated without direct analysis provided by a user. In other embodiments, the diagnostic checks may be implemented by an artificial intelligence algorithm that performs an inference operation on the raw diagnostic data to evaluate the performance of the acoustic coupling article 100 and the ultrasound imaging system 600.

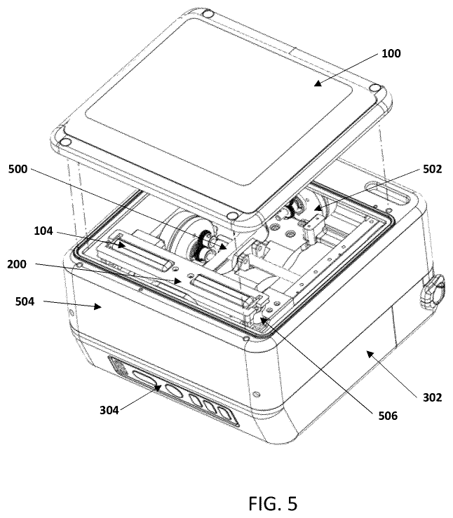

In a preferred embodiment depicted in FIG. 5., the ultrasound probe 302 incorporates a mechanical actuator 500 incorporating one or more motors 502 that translate or rotate one or more ultrasound transducer arrays 104 within a housing body 504 of the ultrasound probe 302. Actuation is controlled by a computer processor in the ultrasound imaging system 600 (or remote from the ultrasound imaging system) that orchestrates the acquisition of position-encoded two-dimensional ultrasound data from varying spatial locations to acquire a volumetric dataset. Further embodiments include elements that apply compressive force 506 to the ultrasound array transducers 104 to ensure consistent contact of the ultrasound array transducers 104 with the surface of the acoustic coupling article 102 and the non-aqueous mobile phase 106. Springs, elastomeric materials, or pneumatic systems are nonlimiting examples of elements that may be integrated in the ultrasound imaging probe 302 for application of a compressive force 504. In another preferred embodiment, the ultrasound probe 302 may incorporate user interface controls 304 and an absorptive reservoir for dispersing a non-aqueous mobile phase 106 that is in contact with an acoustic coupling article 100.

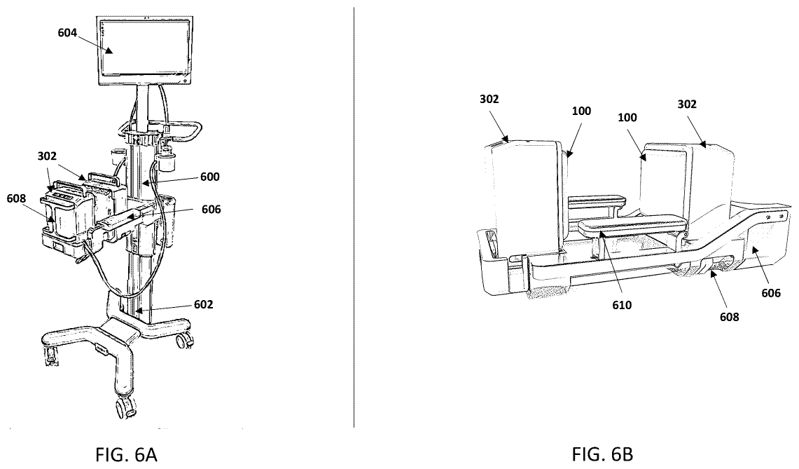

In a preferred embodiment depicted in FIG. 6A, the ultrasound probes 302 and acoustic coupling articles 100 are incorporated in an ultrasound imaging system 600, comprising a wheeled cart 602 and a computer processor and display 604 for acquiring and/or rendering ultrasound images. In a preferred embodiment, the wheeled cart 602 incorporates a support structure 606 that provides predefined storage and operational positions for one or more ultrasound probes 302 and is also capable of supporting a patient's anatomy, such as a limb, while the ultrasound imaging system 600 acquires ultrasound data. In another embodiment, the ultrasound probes 302 incorporate handles 608 and may be detached from the support structure 606 to facilitate handheld operation and handheld placement of the ultrasound probe 302 and acoustic coupling article 100 on a patient's anatomy.

In a preferred embodiment depicted in FIG. 6B, one or more ultrasound probes 302 and affixed acoustic coupling articles 100 are mounted on a support structure 606 that allows for adjustable ultrasound probe 302 positioning relative to a patient's anatomy. One ultrasound probe 302 is maintained in a fixed position, while another ultrasound probe 302 is attached to a linear actuator 608, enabling linear translation towards the fixed-position ultrasound probe 302, thus reducing the gap between the two ultrasound probes 302. When a patient's anatomy, such as a limb, is placed between the ultrasound probes 302, the linear translation of the movable probe causes the acoustic coupling articles 100 on both probes to contact the patient's skin, creating optimal conditions for ultrasound data acquisition by the ultrasound imaging system 600. A support structure 606 may also incorporate an additional support element 610, such as a limb rest, cushion, or pad, to comfortably accommodate the patient's anatomy during the ultrasound scanning process.

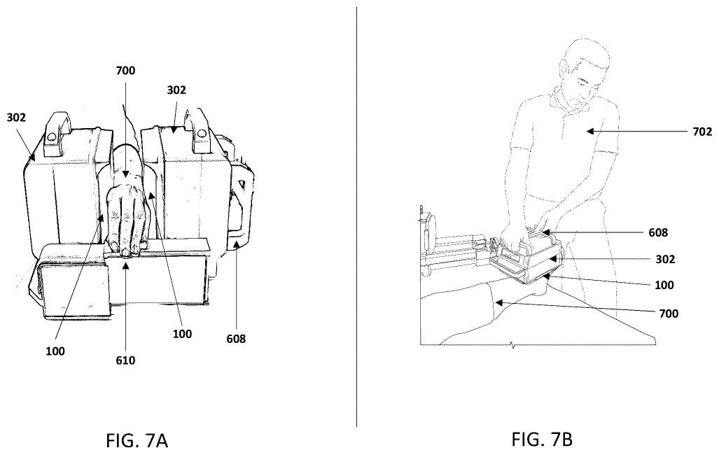

In a preferred embodiment depicted in FIG. 7A, two acoustic coupling articles 100 affixed to two ultrasound probes 302 make contact with a patient's anatomy 700 during an ultrasound scan. The movable ultrasound probe 302 compresses the patient's anatomy 700 to effectuate consistent contact between acoustic coupling article 100 on both ultrasound probes 302 and the patient's anatomy 700. Those skilled in the art will appreciate that this imaging configuration also stabilizes the patient anatomy 700 and reduces anatomical motion which might lead to image degradation or artifacts. An extra liquid coupling phase, such as ultrasound gel or saline, may be necessary at the interface between patient anatomy 700 and the acoustic coupling article 100.

In a preferred embodiment depicted in FIG. 7B, a practitioner 702 utilizes the ultrasound probe 302 by holding onto integrated grips 608 while applying the acoustic coupling article 100 to the patient's anatomy 700. This embodiment permits handheld operation of the ultrasound imaging probe 302 and allows the practitioner to bring the ultrasound imaging probe to the patient, for example if the patient is immobile, on a stretcher, or in a chair.

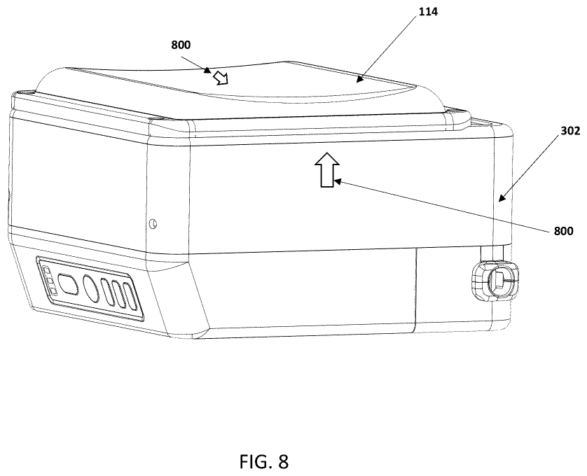

In a preferred embodiment depicted in FIG. 8, the material selected to comprise the exterior layer 114 of the acoustic coupling article 100 is of a malleable composition that can be deformed, thermoformed, or molded into an approximate shape and retains the approximate shape without significant deformation over time. A material with such properties can be simultaneously flexible, to conform to the surface of a patient's anatomy 700, while also rigid enough to enforce the shape of the acoustic coupling surface, be it concave, convex, flat, or another shape. Suitable materials include thermoformable films, such as, but not limited to polyurethanes, polyolefins, and polyether block amide. Additional materials include those that can be cured or cast into a predetermined shape, for example when using a mold, like silicone rubbers and polyurethane rubbers. As may be appreciated by those skilled in the art, the capability to form varied patient contact geometries is advantageous for scanning a wide array of varied patient anatomical structures. Also depicted in FIG. 8 are fiducial markings 800 incorporated on the exterior surface of the acoustic coupling article 114 or the ultrasound probe 302 to aid the user in aligning the patient anatomy with geometry of the ultrasound data acquisition.

Embodiments of the invention also include a computer readable medium comprising one or more computer files comprising a set of computer-executable instructions for performing one or more of the calculations, steps, processes, and operations described and/or depicted herein. In exemplary embodiments, the files may be stored contiguously or non-contiguously on the computer-readable medium. Embodiments may include a computer program product comprising the computer files, either in the form of the computer-readable medium comprising the computer files and, optionally, made available to a consumer through packaging, or alternatively made available to a consumer through electronic distribution. As used in the context of this specification, a “computer-readable medium” is a non-transitory computer-readable medium and includes any kind of computer memory such as floppy disks, conventional hard disks, CD-ROM, Flash ROM, non-volatile ROM, electrically erasable programmable read-only memory (EEPROM), and RAM. In exemplary embodiments, the computer readable medium has a set of instructions stored thereon which, when executed by a processor, cause the processor to perform tasks, based on data stored in the electronic database or memory described herein. The processor may implement this process through any of the procedures discussed in this disclosure or through any equivalent procedure.

In other embodiments of the invention, files comprising the set of computer-executable instructions may be stored in computer-readable memory on a single computer or distributed across multiple computers. A skilled artisan will further appreciate, in light of this disclosure, how the invention can be implemented, in addition to software, using hardware or firmware. As such, as used herein, the operations of the invention can be implemented in a system comprising a combination of software, hardware, or firmware.

Embodiments of this disclosure include one or more computers or devices loaded with a set of the computer-executable instructions described herein. The computers or devices may be a general purpose computer, a special-purpose computer, or other programmable data processing apparatus to produce a particular machine, such that the one or more computers or devices are instructed and configured to carry out the calculations, processes, steps, operations, algorithms, statistical methods, formulas, or computational routines of this disclosure. The computer or device performing the specified calculations, processes, steps, operations, algorithms, statistical methods, formulas, or computational routines of this disclosure may comprise at least one processing element such as a central processing unit (i.e., processor) and a form of computer-readable memory which may include random-access memory (RAM) or read-only memory (ROM). The computer-executable instructions can be embedded in computer hardware or stored in the computer-readable memory such that the computer or device may be directed to perform one or more of the calculations, steps, processes and operations depicted and/or described herein.

Additional embodiments of this disclosure comprise a computer system for carrying out the computer-implemented method of this disclosure. The computer system may comprise a processor for executing the computer-executable instructions, one or more electronic databases containing the data or information described herein, an input/output interface or user interface, and a set of instructions (e.g., software) for carrying out the method. The computer system can include a stand-alone computer, such as a desktop computer, a portable computer, such as a tablet, laptop, PDA, or smartphone, or a set of computers connected through a network including a client-server configuration and one or more database servers. The network may use any suitable network protocol, including IP, UDP, or ICMP, and may be any suitable wired or wireless network including any local area network, wide area network, Internet network, telecommunications network, Wi-Fi enabled network, or Bluetooth enabled network. In one embodiment, the computer system comprises a central computer connected to the internet that has the computer-executable instructions stored in memory that is operably connected to an internal electronic database. The central computer may perform the computer-implemented method based on input and commands received from remote computers through the internet. The central computer may effectively serve as a server and the remote computers may serve as client computers such that the server-client relationship is established, and the client computers issue queries or receive output from the server over a network.

The input/output interfaces may include a graphical user interface (GUI) which may be used in conjunction with the computer-executable code and electronic databases. The graphical user interface may allow a user to perform these tasks through the use of text fields, check boxes, pull-downs, command buttons, and the like. A skilled artisan will appreciate how such graphical features may be implemented for performing the tasks of this disclosure. The user interface may optionally be accessible through a computer connected to the internet. In one embodiment, the user interface is accessible by typing in an internet address through an industry standard web browser and logging into a web page. The user interface may then be operated through a remote computer (client computer) accessing the web page and transmitting queries or receiving output from a server through a network connection.

The present invention has been described with reference to particular embodiments having various features. In light of the disclosure provided above, it will be apparent to those skilled in the art that various modifications and variations can be made in the practice of the present invention without departing from the scope or spirit of the invention. One skilled in the art will recognize that the disclosed features may be used singularly, in any combination, or omitted based on the requirements and specifications of a given application or design. When an embodiment refers to “comprising” certain features, it is to be understood that the embodiments can alternatively “consist of” or “consist essentially of” any one or more of the features. Other embodiments of the invention will be apparent to those skilled in the art from consideration of the specification and practice of the invention.

It is noted that where a range of values is provided in this specification, each value between the upper and lower limits of that range is also specifically disclosed. The upper and lower limits of these smaller ranges may independently be included or excluded in the range as well. The singular forms “a,” “an,” and “the” include plural referents unless the context clearly dictates otherwise. It is intended that the specification and examples be considered as exemplary in nature and that variations that do not depart from the essence of the invention fall within the scope of the invention. Further, all of the references cited in this disclosure are each individually incorporated by reference herein in their entireties and as such are intended to provide an efficient way of supplementing the enabling disclosure of this invention as well as provide background detailing the level of ordinary skill in the art.

As used herein, the term “about” refers to plus or minus 5 units (e.g., percentage) of the stated value.

Reference in the specification to “some embodiments”, “an embodiment”, “one embodiment” or “other embodiments” means that a particular feature, structure, or characteristic described in connection with the embodiments is included in at least some embodiments, but not necessarily all embodiments, of the inventions.

As used herein, the term “substantial” and “substantially” refers to what is easily recognizable to one of ordinary skill in the art.

It is to be understood that the phraseology and terminology employed herein is not to be construed as limiting and are for descriptive purpose only.

It is to be understood that while certain of the illustrations and figure may be close to the right scale, most of the illustrations and figures are not intended to be of the correct scale.

It is to be understood that the details set forth herein do not construe a limitation to an application of the invention.

Furthermore, it is to be understood that the invention can be carried out or practiced in various ways and that the invention can be implemented in embodiments other than the ones outlined in the description above.

1. An acoustic coupling article comprising:
a structural member configured to removably attach to an ultrasound probe, the ultrasound probe comprising at least one ultrasound transducer array;
wherein the acoustic coupling article comprises layers of one or more acoustic transmissive materials capable of conforming to both a surface of a receiving body and to the at least one ultrasound transducer array;
wherein the one or more acoustic transmissive materials is substantially rigid, and wherein the one or more acoustic transmissive materials are capable of restricting motion of the at least one ultrasound transducer array in at least one direction and/or dimension; and
wherein a non-aqueous mobile phase is distributed on a surface of the acoustic coupling article, and wherein the non-aqueous mobile phase provides acoustic coupling between the at least one ultrasound transducer array and the surface of the acoustic coupling article.

2. The acoustic coupling article of claim 1, wherein the total thickness of the acoustic coupling article is between 0.05 cm and 10 cm.

3. The acoustic coupling article of claim 1, wherein the one or more acoustic transmissive materials have Shore hardness greater than Shore OOO 5, a speed of sound between 900 m/s and 2,100 m/s, and an acoustic impedance of less than 3 MRayl.

4. The acoustic coupling article of claim 1, further comprising mechanical retention features, including one or more of grooves, ridges, clips, holes, or threads, that removably mate with the ultrasound probe.

5. The acoustic coupling article of claim 1, further comprising a fluid-tight seal positioned at a joining surface of the acoustic coupling article and the ultrasound probe, the seal comprising a gasket, an O-ring, or a sealant, and structural elements including grooves, mating surfaces, or alignment features.

6. The acoustic coupling article of claim 1, wherein the acoustic coupling article is included in the ultrasound probe or attached to the ultrasound probe for use in volumetric ultrasound imaging.

7. The acoustic coupling article of claim 1, wherein a surface of the acoustic coupling article is substantially flat, providing a linear or planar path of translation or rotation for the at least one ultrasound transducer array.

8. The acoustic coupling article of claim 1, wherein a surface of the acoustic coupling article is curved, wherein the curvature provides a concave, convex, curvilinear, non-linear, or non-planar path of translation for the at least one ultrasound transducer array.

9. The acoustic coupling article of claim 1, wherein the one or more transmissive acoustic materials comprises a thermoplastic material chosen from one or more of polymethylpentene, cross-linked polystyrene and divinylbenzene, polypropylene, polyether block amide, polyester, polyethylene, polyethylene terephthalate, nylon, and polyimide.

10. The acoustic coupling article of claim 1, wherein the one or more transmissive acoustic materials comprise a thermoplastic, a polyurethane, a cured silicone rubber material, or combinations thereof, having a speed of sound between 900 m/s and 2,100 m/s.

11. The acoustic coupling article of claim 1, wherein a surface of an outermost acoustic transmissive material of the layers of one or more acoustic transmissive materials is treated with a surface coating to increase the hydrophilicity, wettability, lubricity, or combinations thereof, of the surface.

12. The acoustic coupling article of claim 1, wherein an outermost acoustic transmissive material of the layers of one or more acoustic transmissive materials is of malleable composition that can be deformed, thermoformed, or molded, into a shape and substantially retains the shape without significant deformation over a one year period of time.

13. The acoustic coupling article of claim 1, wherein an outermost acoustic transmissive material of the layers of one or more acoustic transmissive materials provides a protective barrier layer, configured to shield internal layers of the layers of one or more acoustic transmissive materials from environmental degradation, wherein the protective barrier layer comprises a material that is resistant to one or more of moisture, ultraviolet radiation, chemical exposure, or mechanical damage.

14. The acoustic coupling article of claim 1, wherein the non-aqueous mobile phase comprises a synthetic lubricant, a silicone-based lubricant a petroleum-based lubricant, or combinations thereof.

15. The acoustic coupling article of claim 1, wherein the non-aqueous mobile phase is applied substantially uniformly to a surface of one or more layers of the layers of one or more acoustic transmissive materials that makes contact with the one or more ultrasound transducer arrays.

16. The acoustic coupling article of claim 1, wherein the non-aqueous mobile phase is impregnated into one or more layers of the layers of one or more acoustic transmissive materials, and wherein the non-aqueous mobile phase is released in response to compression or contact forces.

17. The acoustic coupling article of claim 1, further comprising a porous material saturated with the non-aqueous mobile phase, wherein the saturated porous material is applied such that it continuously contacts a surface of the ultrasound probe and at least one non-imaging surface of the at least one ultrasound transducer array, including during translation or rotation of the at least one ultrasound transducer array.

18. An ultrasound imaging and medical instrument guidance system comprising:
an acoustic coupling article for conveyance of ultrasound energy from at least one ultrasound probe to a receiving body;
the at least one ultrasound probe comprising at least one ultrasound transducer array;
one or more computer processors for processing acquired data received from the at least one ultrasound transducer array, and one or more displays for displaying acquired data as an image; and
a non-transitory computer memory comprising instructions that cause the system to translate or rotate the at least one ultrasound transducer array along a surface of the acoustic coupling article to acquire the data at multiple spatial locations.

19. The system of claim 18, wherein the instructions further cause the system to generate a three-dimensional image based on volumetric ultrasound data.

20. The system of claim 18, wherein the instructions further cause the system to detect and report a condition indicating a need for a servicing event of the ultrasound imaging and medical instrument guidance system.

21. The system of claim 18, wherein the instructions further cause the system to perform a calibration sequence to verify proper installation of the acoustic coupling article and data acquisition precision of the at least one ultrasound probe.

22. The system of claim 18, wherein the instructions further cause the system to detect and report an error condition related to insufficient non-aqueous mobile phase on a surface of the acoustic coupling article.

23. The system of claim 18, further comprising a reservoir for holding non-aqueous mobile phase and including a valve designed to release the non-aqueous mobile phase at a location adjacent to the at least one ultrasound transducer array during translation or rotation.

24. The system of claim 18, further comprising a mechanism that applies compressive force to the at least one ultrasound transducer array to maintain contact with a surface of the acoustic coupling article, the mechanism comprising a spring, an elastomeric material, or a pneumatic system.

25. The system of claim 18, further comprising a user interface incorporated on or into the at least one ultrasound probe that provides controls for operating the system, report system state information, reports error conditions, or combinations thereof.

26. The system of claim 18, further comprising one or more ultrasound probes of the at least one ultrasound probe including grips or handles to facilitate handheld operation and positioning of the at least one ultrasound probe on or near a patient's body.

27. The system of claim 18, further comprising fiducial markings on the at least one ultrasound probe and/or on the acoustic coupling article, wherein the fiducial markings indicate a preferred orientation of the at least one ultrasound probe relative to a patient anatomy during ultrasound data acquisition.

28. The system of claim 18, further comprising a wheeled cart to facilitate an alignment of the at least one ultrasound probe with an anatomical scan plane of a patient's anatomy.

29. The system of claim 28, further comprising an ultrasound probe positioning system that compresses a patient's anatomy onto the at least one ultrasound probe and restricts anatomical motion during ultrasound data acquisition.

30. The system of claim 28, wherein the wheeled cart also provides a physical structure for supporting a patient's anatomy during ultrasound data acquisition.

31. The system of claim 18, wherein the acoustic coupling article comprises:
a structural member configured to removably attach to an ultrasound probe, the ultrasound probe comprising at least one ultrasound transducer array;
wherein the acoustic coupling article comprises layers of one or more acoustic transmissive materials capable of conforming to both a surface of a receiving body and to the at least one ultrasound transducer array;
wherein the one or more acoustic transmissive materials is substantially rigid, and wherein the one or more acoustic transmissive materials are capable of restricting motion of the at least one ultrasound transducer array in at least one direction and/or dimension; and
wherein a non-aqueous mobile phase is distributed on a surface of the acoustic coupling article, and wherein the non-aqueous mobile phase provides acoustic coupling between the at least one ultrasound transducer array and the surface of the acoustic coupling article.
